(19) United States
(12) Patent Application Publication
(10) Pub. No.: US 2020/0152299 A1
(43) Pub. Date: May 14, 2020

(54) SYSTEMS AND METHODS FOR REQUESTING AND TRANSMITTING MEDICAL RECORDS BETWEEN MEDICAL PROVIDERS ON UNAFFILIATED MEDICAL DATA NETWORKS

(71) Applicant: MEDICOM TECHNOLOGIES INC., Raleigh, NC (US)

(72) Inventors: MICHAEL ROSENBERG, Raleigh, NC (US); CHASE BALLARD, Raleigh, NC (US); JASON SUTTLES, Raleigh, NC (US); CHRIS WOODLIEF, Raleigh, NC (US)

(21) Appl. No.: 16/681,909

(22) Filed: Nov. 13, 2019

Related U.S. Application Data
(60) Provisional application No. 62/760,111, filed on Nov. 13, 2018.

(51) Int. Cl. G16H 10/60; G16H 80/00; G06Q 50/22

(52) U.S. Cl. CPC G16H 10/60; G06Q 50/22; G16H 80/00

(57) ABSTRACT

The present invention relates, in general, to systems and methods that allow medical providers to request and send medical records, and in particular medical imaging studies, across disparate data exchange networks, and between medical providers that are not part of a same or affiliated data exchange network, thereby eliminating the need for physical media to be created and physically delivered. The present invention further relates to allowing individual patients to transmit medical records to requesting medical providers.

### CROSS-REFERENCE TO RELATED APPLICATION

This application claims the benefit of U.S. Provisional Patent Application No. 62/760,111 entitled “SYSTEM AND METHOD FOR PROVIDING SECURE ACCESS TO A PEER-TO-PEER DATA EXCHANGE NETWORK” filed on Nov. 13, 2018, which is commonly owned, the disclosure of which is incorporated herein by reference in its entirety.

### BACKGROUND

### Field of the Invention

The present invention relates, in general, to systems and methods that allow medical providers to request and send medical records, and in particular medical imaging studies, across disparate data exchange networks, and between medical providers that are not part of a same or affiliated data exchange network, thereby eliminating the need for physical media to be created and physically delivered.

### Description of Related Art

Patients have been carrying their medical images from imaging facilities to referring physicians for as long as medical imaging technology has existed. While recent technological advances in networking and telecommunications have facilitated data sharing across a wide number of industries, this digital revolution has all but bypassed how medical images are shared amongst patients, medical providers, and medical facilities.

The need for timely and secure electronic delivery of medical records, and in particular, medical imaging studies, has never been greater. Traditionally, medical providers wishing to obtain a patient's prior medical imaging data had to rely on hard copies of medical images and diagnostic reports to be created by a previous medical provider, and then physically delivered to the requesting medical providers' location. Hard copies of medical imaging studies are conventionally created using expensive films, or by burning physical media, such as CDs and DVDs. The entire process of creating films, or burning physical media, and shipping the hard copies, can be extremely time-consuming, especially in critical care situations where a patient requires immediate and urgent medical attention.

Furthermore, physical media can oftentimes be in a format that is either unreadable or otherwise provides a barrier to the medical provider who needs to analyze or review the medical images. For example, the delivered hard copy medical images can be stored in a non-standard format, the physical media may not include a native viewer to display the medical images, or the medical images may not be compatible with the medical provider's medical viewing systems or picture archiving and communication systems (PACS). All these issues can cause significant downtime, both from an information technology standpoint and from a patient quality and medical workflow standpoint. There are also inherent risks of viruses and malware that a medical provider must be cognizant of when dealing with CDs and DVDs received from external parties.

Another challenge with physical media is that the medical facilities must find an efficient way to get the medical images from physical media to the medical provider, such as a diagnostic radiologist. In some facilities, technicians manually load medical imaging studies from physical media into a PACS, while other facilities utilize a dedicated workstation specifically designated for reviewing medical imaging studies loaded from external physical media.

Further, medical providers must have a compliant and accurate system in place to label and track physical media, as well as to secure the physical media to prevent inadvertent disclosure and Health Insurance Portability and Accountability Act of 1996 (HIPAA) violations.

Thus, while receiving films, CDs, and DVDs for a patient might appear to be a simple matter, when multiplied by hundreds or thousands of patients, the logistical, compliance, and technical challenges for medical providers become increasingly complex and burdensome. As such, there exists a need for a peer-to-peer communication network that makes it possible for medical records to be electronically delivered in an efficient, secure, streamlined, and auditable manner, instead of the days or weeks currently required through the use of manually burned CDs and DVDs and films, which are physically delivered using postal mail, couriers, and messenger services. Using the present invention, medical records reach their destination immediately upon a request by a recipient in a cost-effective manner.

### SUMMARY

In an embodiment, the invention relates to a method for retrieving a medical record from a remote database, comprising: scheduling a patient for a medical visit by a first medical provider; determining, by the first medical provider, if the patient has a prior medical record located at a second medical provider; determining if the first medical provider and the second medical provider are both registered on a common data exchange network; transmitting, by the first medical provider, an electronic message to the second medical provider if the first medical provider and the second medical provider are not both registered on the data exchange network, wherein the electronic message includes a request for the prior medical record; transmitting, by the first medical provider, a direct message to the second medical provider via the common data exchange network if the first medical provider and the second medical provider are both registered on the common data exchange network, wherein the in-network message includes a request for the prior medical record; uploading, by the second medical provider, the prior medical record, to a secure portal; and transmitting the prior medical record by the second medical provider to the first medical provider via an encrypted peer-to-peer communication channel.

In another embodiment, the invention relates to a method for retrieving a medical record from a remote database, comprising: scheduling a patient for a medical visit by a first medical provider; determining, by the first medical provider, if the patient has a prior medical record located at a second medical provider; transmitting, by the first medical provider, an electronic message to the second medical provider, wherein the electronic message includes a request for the prior medical record, a hyperlink to access a secure portal, and a designated storage location; uploading, by the second medical provider, the prior medical record to a secure portal; transmitting the prior medical record by the second medical provider to the first medical provider via an encrypted peer-to-peer communication channel; and receiving the prior medical record at the first medical provider, wherein the prior medical record is stored at the designated storage location.

In yet another embodiment, the invention relates to a method for retrieving a medical record from a remote database, comprising: scheduling a patient for a medical visit by a first medical provider, wherein the first medical provider has an internal patient record containing internal patient information; determining, by the first medical provider, if the patient has an external medical record located at a second medical provider; transmitting, by the first medical provider, an electronic message to the second medical provider, wherein the electronic message includes a request for the external medical record, a hyperlink to access a secure portal, and a designated storage location; uploading, by the second medical provider, the external medical record to a secure portal, wherein the external medical record contains external patient information; transmitting the external medical record by the second medical provider to the first medical provider via an encrypted peer-to-peer communication channel; receiving the external medical record at the first medical provider, wherein the external medical record stored at the designated storage location; and updating, by the first medical provider, the external patient record to match the internal patient information.

### BRIEF DESCRIPTION OF THE DRAWINGS

These and other embodiments of the disclosure will be discussed with reference to the following exemplary and non-limiting illustrations, in which like elements are numbered similarly, and where:

FIG. 1 is a network architecture diagram of a system for establishing a peer-to-peer communication channel between a diagnostic provider and an external medical provider, according to an embodiment of the invention;

FIG. 2 is a flowchart illustrating the steps of requesting medical reports from an external provider by a diagnostic provider, according to an embodiment of the invention;

FIG. 3 is a flowchart illustrating the steps of receiving and responding to a medical record request by an external provider, according to an embodiment of the invention;

FIG. 4 is a flowchart illustrating the steps of uploading a medical record by an external provider to an uplink portal, according to an embodiment of the invention;

FIG. 5 is a flowchart illustrating the steps of receiving a medical record from an external provider by a diagnostic provider, according to an embodiment of the invention;

FIG. 6 is an exemplary login interface for an uplink portal, according to an embodiment of the invention;

FIG. 7 is an exemplary contact information interface for an uplink portal, according to an embodiment of the invention;

FIG. 8 is an exemplary medical record upload type interface for an uplink portal, according to an embodiment of the invention;

FIG. 9 is an exemplary file browser window for an uplink portal, according to an embodiment of the invention;

FIG. 10 is an exemplary search interface for an uplink portal, according to an embodiment of the invention;

FIG. 11 is an exemplary interface to confirm a medical record selection for an uplink portal, according to an embodiment of the invention; and

FIG. 12 is an exemplary confirmation screen for an uplink portal, according to an embodiment of the invention.

### DEFINITIONS

The following definitions are meant to aid in the description and understanding of the defined terms in the context of the present invention. The definitions are not meant to limit these terms to less than is described throughout this application. Such definitions are meant to encompass grammatical equivalents.

As used herein, the term “medical record” can refer to, for example, any patient data, patient charts, electronic medical records, medical imaging studies, medical and diagnostic images, diagnostic reads and reports, diagnostic notes, medical opinions, medical test and laboratory results, surgical history, family medical histories, medication lists, medical allergies, social histories and habits (such as, for example, drug, tobacco and alcohol use), immunization histories, clinical information, growth and development histories, medical encounter histories, physical examination observations, medical progress notes, fitness and activity data and the like.

As used herein, the term “provider” can refer to, for example, a physician (including, but not limited to, a radiologist, surgeon, primary care physician, and a medical specialist), a physician assistant, a nursing professional, a medical laboratory technician, medical clinics, hospitals, health insurance providers, diagnostic sites, imaging sites, pharmacies, and the like. A “provider” can further be an individual patient, an academic institution, a government research laboratory, a non-profit entity, or a for-profit entity, such as a pharmaceutical, health insurance, biotechnology, wearable device, physiological monitoring, or medical device company.

### DETAILED DESCRIPTION

It should be understood that aspects of the invention are described herein with reference to the figures, which show illustrative embodiments. The illustrative embodiments herein are not necessarily intended to show all embodiments in accordance with the invention, but rather are used to describe a few illustrative embodiments. Thus, aspects of the invention are not intended to be construed narrowly in view of the illustrative embodiments. In addition, although the invention is described with respect to its application for the transfer of medical information containing medical imaging studies in the Digital Imaging and Communications in Medicine (DICOM) format, it is understood that the system could be implemented in any setting where the transfer of any type or form of medical or patient data may be useful.

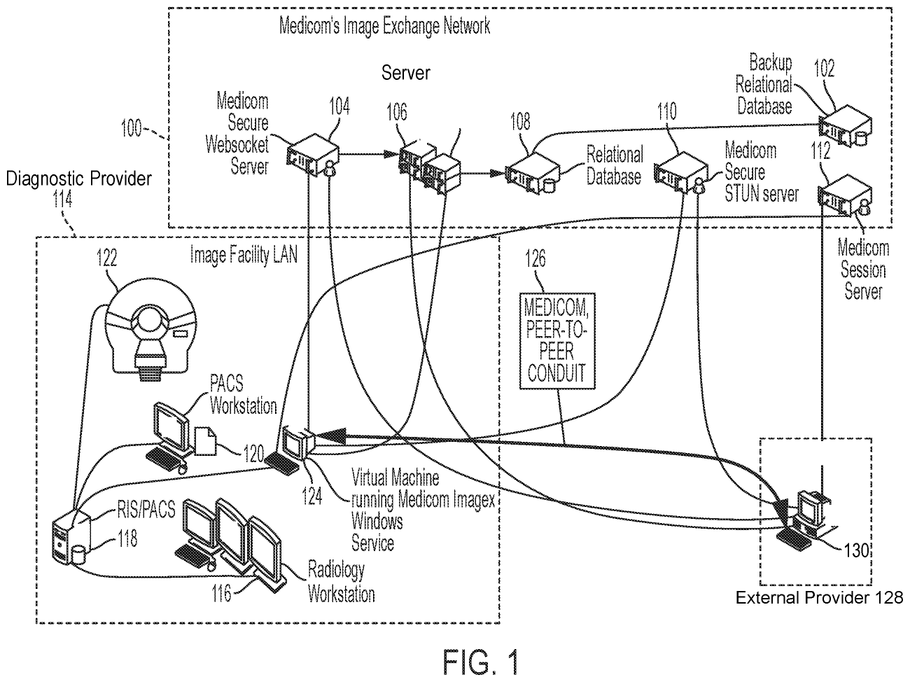

FIG. 1 is a network architecture diagram of a system for establishing a peer-to-peer communication channel between a diagnostic provider 114 and an external provider 128, according to an embodiment of the present invention. The data exchange network 100 include various components, such as a backup relational database 102, a web socket server 104, a server 106, a relational database 108, a secure STUN (Session Traversal of User Datagram Protocol [UPD] Through Network Address Translators [NATs]) server 110, and a session server 112.

The data exchange network 100 is preferably a distributed, public access network, such as the Internet, wherein an external provider device 130 and the web socket server 104 are capable of interacting with and through the data exchange network 100 using various protocols such as Transmission Control Protocol/Internet Protocol (TCP/IP), Hypertext Transport Protocol (HTTP), and File Transfer Protocol (FTP). However, those of ordinary skill in the art will appreciate that the data exchange network 100 is not limited thereto. The servers described herein can include a transmitter, receiver, transceiver, and/or any other communication interface that allows the servers to bi-directionally communication with other devices, components, and servers on the data exchange network 100 as well as located outside of the data exchange network 100. The servers can also include a local storage, central processing unit (CPU), and a file system. In yet another embodiment, the servers can be a cloud-based computing and storage network.

More specifically, the data exchange network 100 may be any type of network suitable to allow interaction between the external provider device 130 and the web socket server 104. For example, the data exchange network 100 may be a wired network, a wireless network, or any combination thereof. Further, the data exchange network 100 may include a distributed computing network, an intranet, a short-range mesh network, a local-area network (LAN) and/or a wide-area network (WAN), or any combination thereof. The data exchange network 100 may be comprised of wired and wireless elements. For example, the LAN may make use of wifi in its many variations and the WAN may make use of cellular networks using technologies including, but not limited to, CDPD, CDMA, GSM, PDC, PHS, TDMA, FLEX, ReFLEX, iDEN, TETRA, DECT, DataTAC, Mobitex, EDGE and other 2G, 3G, 4G and LTE technologies.

The data exchange network 100 is communicatively coupled to the external provider 128. The external provider 128 can include the external provider device 130, which can be a PACS, a PACS workstation, a database, a server, and the like. In a preferred embodiment, the external provider device 130 is a PACS workstation that allows an external provider to view medical record requests, messages, notifications, requests from external parties, etc., and which allows the external provider to perform various actions such as responding to such medical record requests.

The data exchange network 100 is further communicatively coupled to a diagnostic provider 114. The diagnostic provider 114 can have multiple devices, such as provider workstations 116, a PACS 118, a PACS workstation 120, and imaging equipment 122. In an embodiment, the diagnostic provider 114 can provide diagnostic imaging, interventional radiology procedures, and diagnostic reads and interpretations of medical imaging studies.

The imaging equipment 122 may include, but is not limited to, diagnostic and imaging equipment used for computed tomography (CT), fluoroscopy, magnetic resonance imaging (MRI), magnetic resonance angiography (MRA), mammography, nuclear medicine, x-ray, positron emission tomography (PET), elastography, photo-acoustic imaging, echocardiograph and ultrasound studies.

At the diagnostic provider 114, the imaging equipment 122, PACS workstation 120, and provider workstations 116 are all communicatively coupled to the PACS 118 via the LAN. In addition, a virtual machine 124 running the data exchange network service is coupled to the PACS 114. The virtual machine 124 is further coupled to the data exchange network 100 via a network connection, and the virtual machine 124 can be coupled to the external provider device 130 via a secure peer-to-peer communication channel 126.

In an embodiment, the virtual machine 124 is further communicatively coupled to the server 106, allowing the diagnostic provider 114 to access various computing functionality of the server 106.

The PACS 118 is a medical imaging technology that provides economical storage and convenient access to images from multiple modalities (i.e., imaging source machine types). Electronic images and reports are transmitted digitally via PACS 118; this eliminates the need to manually file, retrieve, or transport film jackets. The universal format for PACS image storage and transfer is DICOM. Non-image data, such as scanned documents, may be incorporated using consumer industry-standard formats like Portable Document Format (PDF) and Clinical Document Architecture (CDA or C-CDA), once encapsulated in DICOM.

The server 106 is configured to serve a web-based interface to a display on the external provider device 130. The server 106 can include a combination of integrated software components and an allocation of computational resources, such as memory, multiple nodes, and processes on these nodes for executing the integrated software components on at least one processor, the combination of the software and computational resources being dedicated to performing a particular function on behalf of the external provider device 130. Resources from multiple nodes in the multi-node environment within the data exchange network 100 can be allocated to run software on the server 106. A particular combination of the software on a node and the allocation of the resources from the server 106 is referred to herein as a server instance or instance. Thus, the server 106 can comprise multiple server instances that can run on multiple nodes. Several instances of the server 106 can even run on the same node.

The web socket server 104 can verify connections on the data exchange network 100 using the relational database 108. The relational database 108 can be, for example, a MySQL relational database. The web socket server 104 can verify connections by referencing the relational database 108 comprising of entities (such as, for example, individuals, groups, medical providers, diagnostic providers, etc.) involved in, for example, a patient's continuum of care.

The web socket server 104 is communicatively coupled to a STUN server 110. The STUN server 110 allows the external provider device 130 to determine its public address, the type of NAT the external provider device 130 is located behind, and the internet-side port associated by the NAT with a particular local port. The STUN server 110 can be internal to the data exchange network 100, and can be hosted by the data exchange network 100. Alternatively, the STUN server 110 can be located externally to the data exchange network 100. In an embodiment, the STUN server 110 can be a public sever operated by a third-party. This information is used to set up UDP communication between the external provider device 130 and a VoIP provider to establish a call between the two locations. The STUN protocol is defined in RFC 3489. In response to receiving the request for a networking address of the external provider device 130, the STUN server 110 operates to determine the public networking address of the external provider device 130.

The web socket server 104 does not inherently retain the state of past requests from the external provider device 130. To solve this problem, the application workflow is maintained by the session server 112, which bundles and stores session data related to accesses from any system node or device. Each time an external provider device 130 enters the data exchange network 100, the external provider device 130 can be given a new unique session ID. Session data representing a state of the external provider device 130 is stored in a memory in the session server 112. As each subsequent request associated with the external provider device 130 is received, the session data is retrieved from the session server 112 using the session ID. The subsequent request is then processed using the session data to provide a web-based interface to the external provider device 130. The session server 112 stores session data unique to each user device session accumulated over time, and over multiple web connections and instances.

The peer-to-peer communication channel 126 has been fully described in commonly owned U.S. patent application Ser. No. 15/361,319, filed on Nov. 25, 2016, and entitled, “METHODS AND SYSTEMS FOR PROVIDING SECURE AND AUDITABLE TRANSFER OF ENCRYPTED DATA BETWEEN REMOTE LOCATIONS”, which is hereby incorporated by reference in its entirety.

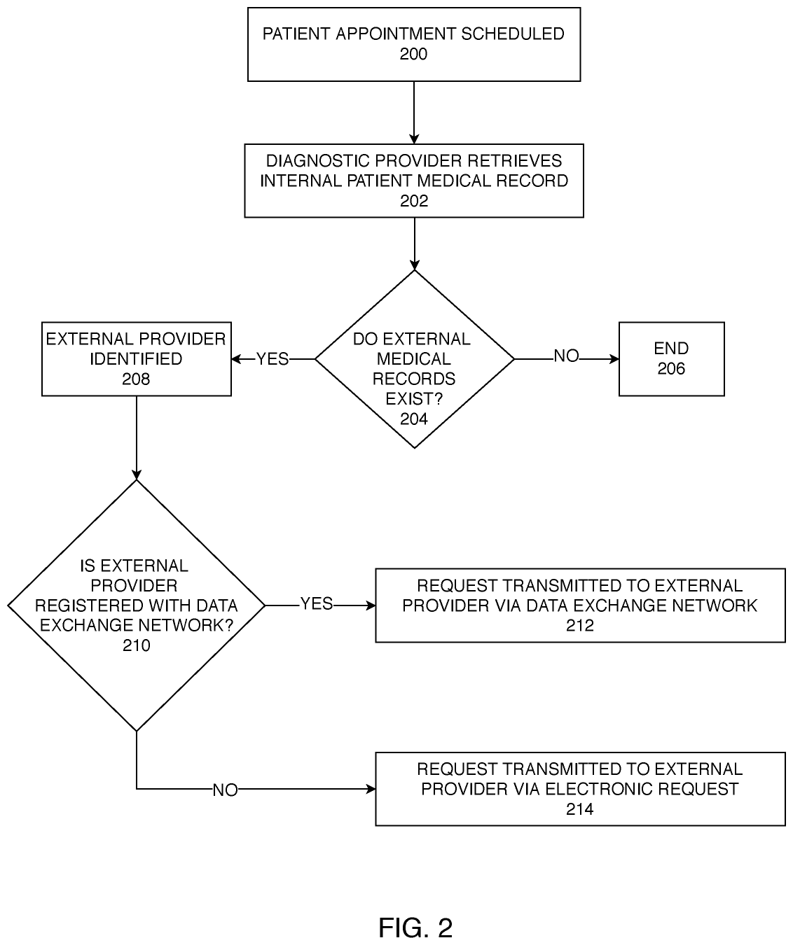

FIG. 2 is a flowchart illustrating the steps of requesting medical reports from an external provider 128 by a diagnostic provider 114, according to an embodiment of the invention. At step 200, a patient schedules an appointment with the diagnostic provider 114. In an embodiment, the patient can utilize patient portal software managed, operated, or provided by the diagnostic provider 114. The patient portal software can be an online website, or mobile application, that is connected to the patient's electronic health records, and which is centrally focused on providing patient access to health data. The patient portal software can allow patients to access lab results, physician notes, their health histories, discharge summaries, immunizations, and the like, as well as schedule medical consultations, visits, and procedures. The patient portal software can be, for example, MyChart® developed by Epic Systems Corporation, the Patient Portal and Healow® App developed by eClinicalWorks LLC, the athenaCommunicator® developed by AthenaHealth, Inc., and similar patient portal software developed by Accenture Federal Services plc, Allscripts Healthcare Solutions, Inc., Cerner Corporation, Computer Programs and Systems Inc., InteliChart LLC, and the like.

In an embodiment, after scheduling the appointment via the patient portal software, the patient can select the appointment and provide additional details or notes to the diagnostic provider 114, as well as can upload external medical records from an external provider 128.

At step 202, the diagnostic provider 114 retrieves an internal medical record for the patient. The internal medical record can be stored on the PACS 118, PACS workstation 120, and/or another database operated, managed, or owned by diagnostic provider 114. In an embodiment, the internal medical record can be stored on a third-party database to which the diagnostic provider 114 has access to.

In an embodiment, the internal medical record for the patient can be stored on distributed databases, where medical images can be stored at one location, and diagnostic reads stored at another location.

At step 204, the server 106 determines if the patient has any external medical records that are not included in the internal medical record, where the external medical records are related to the visits by the patient to external providers. In an embodiment, the server 106 analyzes the internal medical record and determines if the internal medical record contains references, notes, or other indications of prior patient visits to any external providers.

In another embodiment, the diagnostic provider 114 can physically ask the patient if the patient has any prior visits to any external providers. In yet another embodiment, the patient can upload external medical records directly via the patient portal software.

If the server 106 determines that the patient does not have any external medical records not included in the internal medical records, the process ends at step 206. If, however, the server 106 determines that the patient has an external medical record not included in the internal medical record, then at step 208, the server 106 identifies at least one external provider associated with the external medical record.

At step 210, the server 106 determines if the external provider 128 is registered with the data exchange network 100. If the external provider 128 is registered with the data exchange network 100, then at step 212, the diagnostic provider 114 can transmit a direct message to the external provider 128 via the data exchange network 100 requesting the external medical record.

In an embodiment, the server 106 can compare an identifier associated with the external provider 128 against a database, such as the relational database 108, to determine if the external provider 128 is registered with the data exchange network 100. The identifier can be, but is not limited to, a provider name, an address, a telephone number, an e-mail address, a facsimile number, an associated physician name, a facility identification number, a National Provider Identifier (NPI), a Medicare provider number, a CMS Certification Number (CCN), and the like.

If, however, the server 106 determines that the external provider 128 is not registered with the data exchange network 100, then at step 214, the external provider 128 can transmit an electronic request to the external provider 128. In an embodiment, the electronic request can include, but is not limited to, an electronic facsimile (e-fax), secure electronic mail (e-mail), messaging via the HL7 format, direct message via a Health Information Service Provider (HISP), and the like. In a preferred embodiment, the electronic request is in the form of an e-fax.

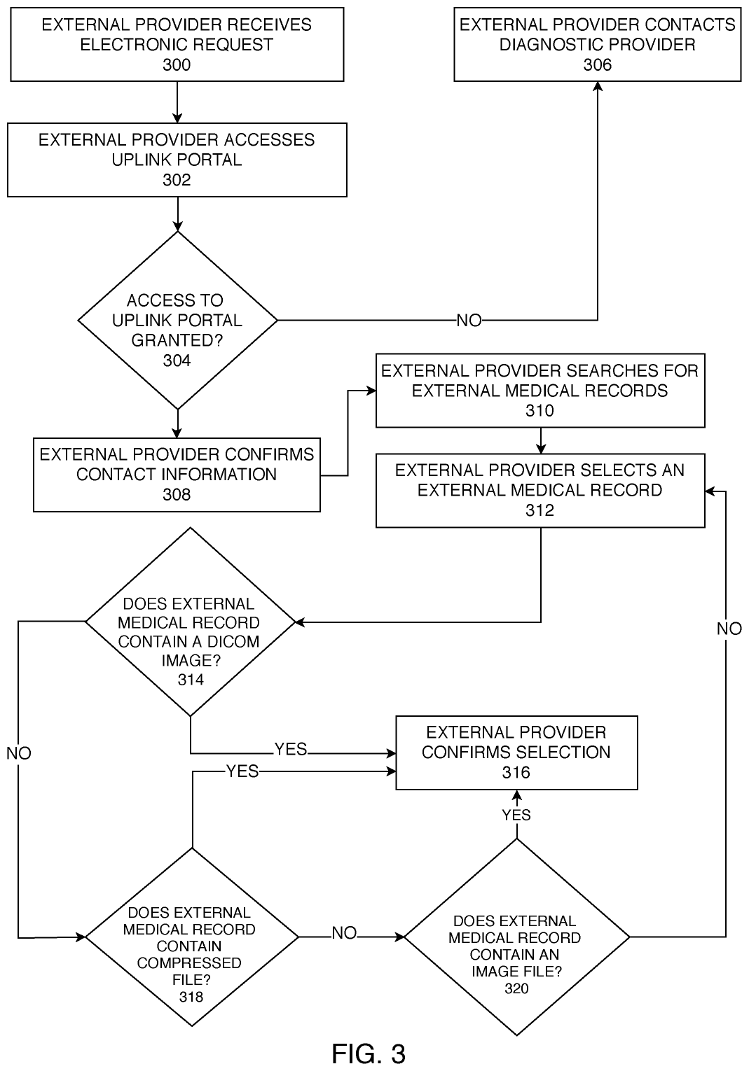

FIG. 3 is a flowchart illustrating the steps of receiving and responding to a medical record request by an external provider 128, according to an embodiment of the invention. At step 300, the external provider 128 receives the electronic request for medical records for the patient. As discussed herein, the electronic request can be in the form of an e-fax.

In an embodiment, the electronic request contains a link that allows the external provider 128 to access an uplink portal. The uplink portal can be managed, developed, or otherwise provided by the data exchange network 100. The uplink portal can be a secure website where the external provider 128 can upload medical records for secure transmission to the diagnostic provider 114.

The link can be a hyperlink to a secure web address, a secure private network address, a short URL, and the like. In another embodiment, the link is in the form of readable code, such as a standard barcode or a matrix barcode, such as a Quick Response (QR) code, Snapcode, two-dimensional barcode, three-dimensional barcode, SnapTag, SPARQCode, and the like. In this embodiment, the external provider 128 can scan the readable code via a mobile device, and can subsequently access the uplink portal via a browser on the mobile device.

In an embodiment, the link can be made available for a limited duration to the external provider 128. After expiry of this duration, the link is no longer valid, and cannot be used to access the uplink portal. Either the diagnostic provider 114 or the server 106 can set a duration for the link. In an embodiment, the duration of the link can be set to a pre-defined time from the time of transmission of the link to the external provider 128. For example, the link can expire or become invalid 72 hours from transmission to the external provider 128.

In another embodiment, the duration of the link can be set to a pre-defined time after being accessed by the external provider 128. For example, the link can expire or become invalid 72 hours after the external provider 128 initially accesses the link.

In another embodiment, the link be configured for “one-time” use, such that once the external provider 128 selects the link, it will no longer be valid and useable for subsequent access to the uplink portal.

In an embodiment, the server 106 generates the link using a hashing algorithm, such as an MD5 message-digest algorithm. The hashing algorithm can utilize an address of the external provider 128 as an input, such as their e-mail address, direct messaging address, secure messaging address, IP address, and the like. In addition, the hashing algorithm can also utilize a timestamp as an input.

In yet another embodiment, the link can be displayed on the external provider device 130 for a limited duration. After expiration of this duration, the link is no longer visible or valid. In one aspect, an application, such an e-mail application or web browser used to access e-mail, can effectively “erase” the link, similar to how social media posts and messages in the social application Snapchat disappear from view after a certain period of time.

In yet another embodiment, the electronic request can include not only a link but also credentials for the external provider 128 to supply to the uplink portal to gain access to the uplink portal. In an embodiment, the credentials can include, but is not limited to, a username, password, unique identifier, access code, or a combination thereof. The credentials can be generated by the diagnostic provider 114 or the server 106. The credentials can randomly generated, can be valid for a “one-time” use, and the like. In an embodiment, the external provider 128 can be prompted to enter their e-mail address as a credential.

At step 302, the external provider 128 selects the link to access the uplink portal. In an embodiment, the uplink portal is accessed by the external provider 128 via a native web browser launched on the external provider device 130. In an embodiment, the data exchange network 100 is configured to initiate the launch of a lightweight web browser upon selection of the hyperlink by the external provider 128. The lightweight web browser is configured to load and display the uplink portal.

In an embodiment, the link directs the external provider 128 to a network or Internet address which automatically initiates a download a software file from the server 106 that contains executable software to launch the uplink portal. The external provider 128 may be prompted to accept the initiation of the software file download. Once the software file is downloaded to the external provider device 130, the external provider 128 can be prompted through installation steps for the software file.

In an embodiment, upon receiving a subsequent electronic request for external medical records, the link automatically launches the uplink portal that has previously been installed on the external provider device 130 upon selection of the link by the external provider 128.

In yet another embodiment, the uplink portal can be integrated with a PACS or electronic medical record system utilized by the external provider 128 (collectively, “the external provider PACS”). For example, the uplink portal can be integrated via an application programming interface (API). In this embodiment, the external provider 128 can receive the electronic request via a secure e-mail, a secure message via the HL7 format, a direct message, a chat message, and the like on the external provider PACS. The external provider PACS can include, for example, electronic medical record software developed by Epic Systems Corporation.

At the 304, the server 106 determines if the external provider 128 has been granted access to the uplink portal. This step prevents misuse of the uplink portal, or intentional or unintentional unauthorized access by third-parties which may obtain the link. In an embodiment, either the diagnostic provider 114, or the server 106, can grant access to the uplink portal to the external provider 128, and can further designate additional authorizes third-parties who may access the link.

In an embodiment, the server 106 can determine if the external provider 128 has access to the uplink portal by analyzing an Internet protocol address associated with the external provider device 130. In another embodiment, the external provider 128 can be prompted to input credentials on a login interface for the uplink portal.

If the server 106 determines that the external provider 128 does not have access to the uplink portal, then at step 306, the external provider 128 is prompted to contact the diagnostic provider 114 or the data exchange network 100 administrator.

If, however, the server 106 determines that the external provider 128 does have access to the uplink portal, then at step 308, the external provider 128 is prompted to enter their contact information. The contact information can include, but is not limited to, a provider name, an address, a telephone number, an e-mail address, a facsimile number, an associated physician name, a facility identification number, an NPI, a Medicare provider number, a CCN, and the like. In an embodiment, the contact information for the individual accessing the uplink portal at the external provider 128 can also be entered. The individual can be, for example, a physician, a medical record administrator, an office manager, a physician assistant, a nurse, a bookkeeper, a receptionist, and the like affiliated with, or working with, the external provider 128.

At step 310, the external provider 128 can browse, search, and view medical records that are stored on an external provider database. In an embodiment, the external provider database can be located on the external provider device 130, and/or another device or server operated, managed, or owned by external provider 128. In an embodiment, the external provider database can operated or managed by a third-party administrator to which the external provider 128 has a contractual relationship.

In another embodiment, the external provider database consists of distributed databases, where medical images can be stored at one location, and diagnostic reads stored at another location.

In an embodiment, the uplink portal is configured to have read-only access to the external provider database, and can display the external medical records on an interface on the uplink portal.

In an embodiment, the uplink portal allows the external provider 128 to search and filter external medical records based on criteria such as a patient age or age range, modality, imaging center, referring physician, date, and the like. In an embodiment, the external provider 128 can search for a specific patient using criteria such as a first name, middle name, last name, gender, date of birth, a social security number, an address, a telephone number, a medical record number, or any other unique character string related to the patient whose records are being requested by the diagnostic provider 114. In an embodiment, the external provider 128 can further search and filter external medical records based on a visit description, insurance code, study date, and the like.

In an embodiment, external medical records associated with the requested patient are automatically located by the server 106, and displayed on the uplink portal. In this embodiment, the server 106 can associate the link with the patient-specific information in the relational database 108. Upon accessing the uplink portal by the external provider 128, the server 106 can utilize the patient-specific information to search the external provider database. For example, the patient-specific information, can include, for example, a first name, middle name, last name, gender, date of birth, a social security number, an address, a telephone number, a medical record number, or any other unique character string related to the patient whose records are being requested by the diagnostic provider 114.

At step 312, the external provider 128 can then select the appropriate medical record or records for the requested patient. In an embodiment, the external provider 128 can be prompted to upload the selected external medical record from the external provider database to the uplink portal. In this embodiment, the uplink portal provides a file browser window where the external provider 128 can select a file, multiple files, a folder, and/or multiple folders containing external medical record.

In an embodiment, the external provider 128 can receive a direct request from the diagnostic provider 114 to upload medical records, for example via e-mail, a hard copy request or a copy-and-pasted requested directly into the external provider 128 electronic medical records software, patient portal software, or PACS.

In an embodiment, the external provider 128 can drag-and-drop files containing the selected medical record for the requested patient onto a dialogue window on the uplink portal.

In yet another embodiment, the external provider 128 can upload an attachment, such as a file or folder, to the selected external medical record. In this embodiment, the additional files, which can be, for example, medical images which are not be stored with the external medical record at the external provider database. The medical images may be stored separately in another storage location and can be separately uploaded by the external provider 128, along with the selected external medical record.

At step 314, the server 106 analyzes the selected external medical record to determine if the medical record contains any DICOM images. If the selected external medical record contains a DICOM image, then the process continues to step 316, where the external provider 128 is prompted to confirm the selection of the external medical record.

If, however, the selected external medical record does not contain any DICOM images, then at step 318, the server 106 analyzes the selected external medical record to determine if the medical record contains any compressed files, such as files in a Zip, 7Z, RAR, gZip, Tar, Shar, ACE, ARC, ARJ, DMG, LHZ, LZX, MPQ, PEA, rZip, SQX, UDA, Xar, ZPAQ, and the like, compression and/or archiving format. In a preferred embodiment, the server 106 analyzes the selected external medical record to determine if any files in the Zip format are contained therein. If the selected external medical record contains a compressed file, then the process continues to step 316, where the external provider 128 is prompted to confirm the selection of the external medical record.

If, however, the selected external medical record does not contain any compressed files, then at step 320, the server 106 analyzes the selected external medical record to determine if the medical record contains any image files, such as files in the JPEG, Bitmap, TIFF, GIF, PNG, Raw, encapsulated PDF, and the like, format. If the selected external medical record contains an image file, then the process continues to step 316, where the external provider 128 is prompted to confirm the selection.

If the selected external medical record does not contain any image files, then the process continues to step 310, where the external provider 128 is prompted to select another external medical record for the requested patient.

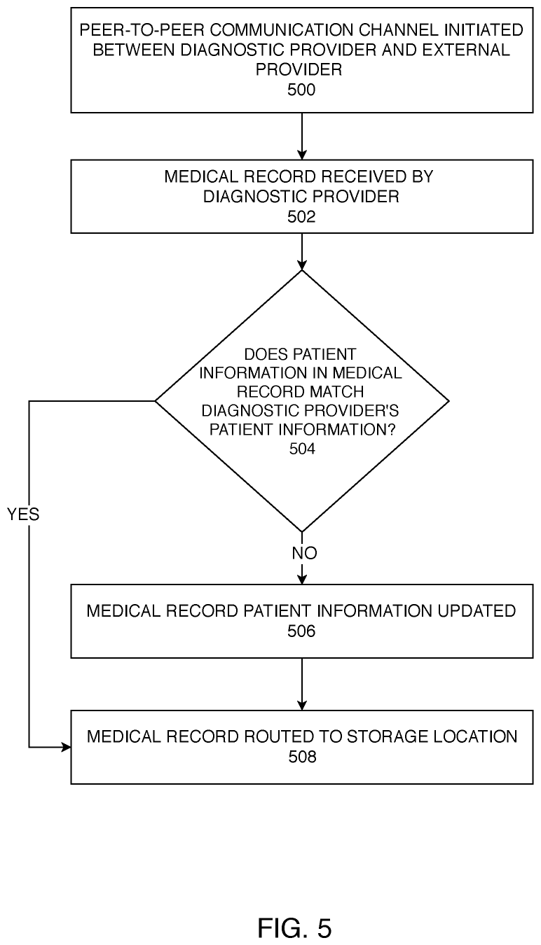

Once the external provider 128 confirms the selected external medical record, as well as any uploaded and attached files, the server 106 initiates the secure peer-to-peer communication channel directly between the external provider 128 and the diagnostic provider 114 so that the selected external medical record can be transmitted to the diagnostic provider 114, as discussed in more detail herein in FIG. 5. In an embodiment, the external provider 128 can remove or delete the selected external medical record, in the event the external provider 128 does not confirm the selection of the external medical record.

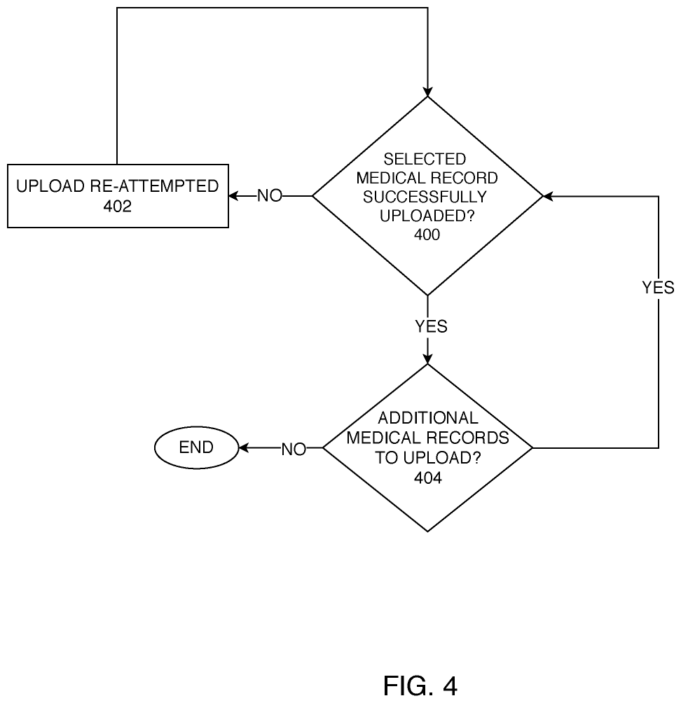

FIG. 4 is a flowchart illustrating the steps of uploading a medical record by an external provider 128 to an uplink portal, according to an embodiment of the invention. In an optional step 400, after the external provider 128 has confirmed the selected external medical record at step 316 shown in FIG. 3, the server 106 determines if the selected external medical record has been successfully uploaded to the uplink portal. In an embodiment, the server 106 can determine if the selected external medical record is corrupt, incomplete, or otherwise has any flaws or inconsistencies which would prevent the selected external medical record from being transmitted to, or accessed in its entirety by, the diagnostic provider 114.

In another embodiment, the uplink portal can provide a preview of the selected external medical record to the external provider 128. The external provider 128 can manually review the selected external medical record to ensure that it is not corrupt, incomplete, or otherwise has any flaws or inconsistencies which would prevent the selected external medical record from being transmitted to, or accessed in its entirety by, the diagnostic provider 114. In an embodiment, the external provider 128 is prompted to confirm that the patient information of the selected external medical record matches the patient whose records are being requested by the diagnostic provider 114.

If the selected external medical record was not successfully uploaded to the uplink portal, then at step 402, the external provider 128 is prompted to re-upload the selected external medical record. The process then continues to step 400 where the server 106 can again determine if the selected external medical record was successfully uploaded to the uplink portal.

If, however, the selected external medical record was successfully uploaded to the uplink portal, then at step 404, the external provider 128 is asked if they would like to upload additional medical records. If so, the external provider 128 can then select an additional medical record or records for the requested patient, and the process continues to step 400 where the server 106 determines if the selected external medical record has been successfully uploaded to the uplink portal. If the external provider 128 does not wish to upload additional medical records, then the process ends.

FIG. 5 is a flowchart illustrating the steps of receiving a medical record from an external provider 128 by a diagnostic provider 114, according to an embodiment of the invention. At step 500, the server 106 initiates the secure peer-to-peer communication channel directly between the external provider 128 and the diagnostic provider 114. The peer-to-peer communication channel is not coupled to the server 106, or to any third-party, external, or internal servers, computing devices, machines, cloud networks, and the like. The peer-to-peer communication channel allows for the external medical record to be securely transmitted directly from the external provider 128 to the diagnostic provider 114 without any intermediary parties or systems coming into contact with the external medical record while in transit via the peer-to-peer communication channel. The external medical record is not downloaded to the server 106, or to any third-party server, for subsequent retransmission to the diagnostic provider 114.

In an embodiment, the server 106 can encrypt the external medical record prior to transmission by utilizing cryptographic protocols, hashing algorithms, key-based encryption, obfuscation, and the like. In this embodiment, the diagnostic provider 114 can be provided with a private key to decrypt the encrypted external medical record, where the private key can be transmitted along with the external medical record, or alternatively, can be communicated in a separate transmission via the peer-to-peer communication channel.

In an embodiment, the server 106 can compress the external medical record prior to transmission. The compression can be lossless such that the original data in the external medical record can be fully reconstructed from a compressed state upon receipt by the diagnostic provider 114.

At step 502, the external medical record is received by the diagnostic provider 114. In an embodiment, the external medical record is stored at a storage location designated by the diagnostic provider 114. For example, upon initially requesting the external medical record, the diagnostic provider 114 can specify a location to store the external medical record upon receipt from the external provider 128. In an embodiment, the address of the storage location is saved by the server 106 in the relational database 108.

Upon initiating the peer-to-peer communication channel, the server 106 retrieves the address of the storage location and configures the peer-to-peer communication channel to automatically route the external medical record to the storage location upon receipt by the diagnostic provider 114.

In an embodiment, multiple storage locations for various components of the external medical record can be designated by the diagnostic provider 114. For example, DICOM images in the external medical record can be stored at the PACS 118, while a medical report in the external medical record can be stored at PACS workstation 120. Such storage locations are intended for illustrative purposes only, and the diagnostic provider 114 may designate various internal, external, and/or distributed databases and storage locations at which to store various components of the external medical record.

In another embodiment, the server 106 can automatically classify various components of the external medical record prior to transmission by the external provider 128, or after receipt by the diagnostic provider 114. The server 106 can then select appropriate storage locations for the various components of the external medical record based on the determined classifications.

In yet another embodiment, the external medical record can be placed in a checkpoint upon receipt by the diagnostic provider 114, instead of being routed to a designated storage location. The diagnostic provider 114 can manually review the external medical record and manually specify a storage location after review in the checkpoint.

At step 504, the server 106 analyzes the external medical record to determine if the patient information in the external medical record matches the internal patient information for the patient. In an embodiment, the server 106 determines if there are any patient information discrepancies between the external medical record and the internal patient information. The patient information can include, but not limited to, a date of birth, a first name, a middle name, a last name, a social security number, an address, a telephone number, an e-mail address, a medical record number, a patient identification number, Personally identifiable information (PII), Protected Health Information (PHI), and the like.

If the server 106 determines an inconsistency in the patient information in the external medical record, then at step 506, the inconsistent patient information in the external medical record is updated, replaced, or otherwise modified to match the internal patient information. For example, if the external medical record has a patient identification number native to the external provider 128, then the server 106 replaces the patient identification number to match the patient identification number native to the diagnostic provider 114. The process continues to step 508 where the external medical record is then routed to the designated storage location or locations.

In an embodiment, metadata, headers, and the like containing inconsistent patient information in the external medical record can be updated, replaced, or otherwise modified with internal patient information.

If, however, the server 106 determines that no inconsistencies exist in the patient information in the external medical record, then the external medical record is routed to the designated storage location or locations at step 508.

In an embodiment, the diagnostic provider 114 may specify that the electronic request is a stat or urgent request for the external medical record. In this embodiment, the upon receipt of the external medical record by the diagnostic provider 114, the external medical record can instantly be displayed on the provider workstation 116 or the PACS workstation 120, or can be routed directly to a device utilized by a requesting individual at the diagnostic provider 114, such as a requesting radiologist.

In yet another embodiment, the external medical record can be displayed in real-time as data packets for the external medical record are received by the diagnostic provider 114. For example, if the external medical record contains a DICOM image, the DICOM image can be streamed to the provider workstation 116 or the PACS workstation 120 as the DICOM image is being received from the external provider 128.

In an embodiment, the server 106 can store an audit trail related to the electronic request. The audit trail can include various information, such as details related to the diagnostic provider 114 and the external provider 128, as well as the electronic request date, requested patient information, technical information related to the external provider device 130, transmission information related to the peer-to-peer communication channel (i.e., transmission time, speed, latency, and the like), storage location information at the diagnostic provider 114, contact information of the external provider 128, and the like.

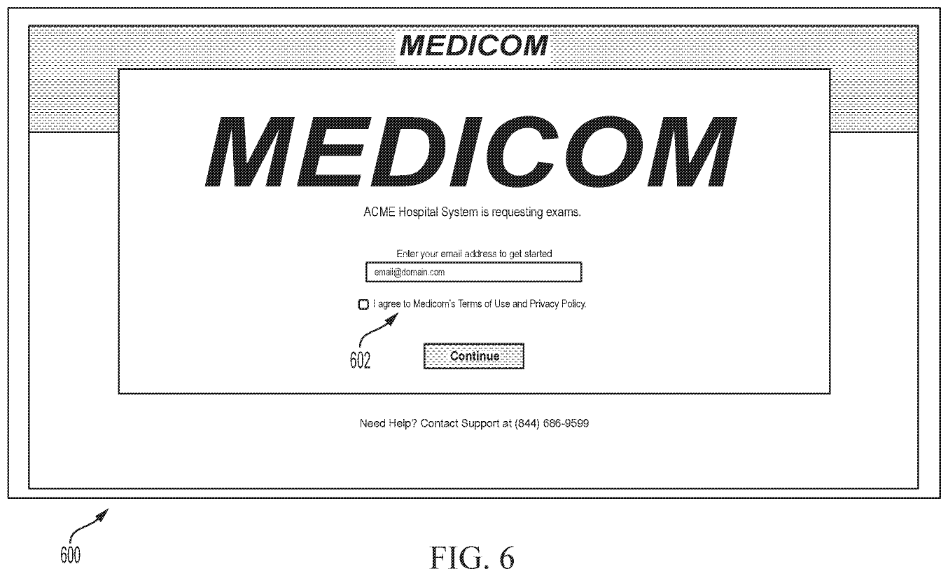

FIG. 6 is an exemplary login interface 602 for an uplink portal 600, according to an embodiment of the invention. Upon accessing the link in the electronic request by the external provider 128, the uplink portal 600 is launched on the external provider device 130, as described herein. In an embodiment, the login interface 602 allows the external provider 128 to input credentials, such as a username, password, unique identifier, access code, or a combination thereof. In a preferred embodiment, external provider 128 can be prompted to enter their e-mail address as a credential, as shown in FIG. 6.

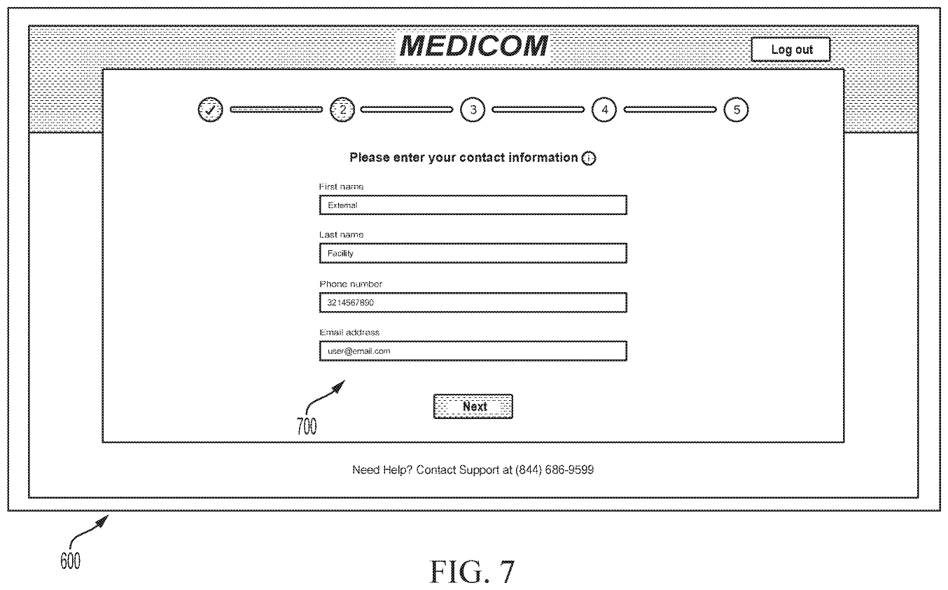

FIG. 7 is an exemplary contact information interface 700 for an uplink portal 600, according to an embodiment of the invention. The contact information interface 700 allows the external provider 128 to enter their contact information, such as, but not limited to a provider name, an address, a telephone number, an e-mail address, a facsimile number, an associated physician name, a facility identification number, an NPI, a Medicare provider number, a CCN, and the like. In an embodiment, the contact information for the individual accessing the uplink portal at the external provider 128 can also be entered. The individual can be, for example, a physician, a medical record administrator, an office manager, a physician assistant, a nurse, a bookkeeper, a receptionist, and the like.

In an embodiment, the contact information entered by the external provider 128 can be stored by the server 106 as part of an audit trail.

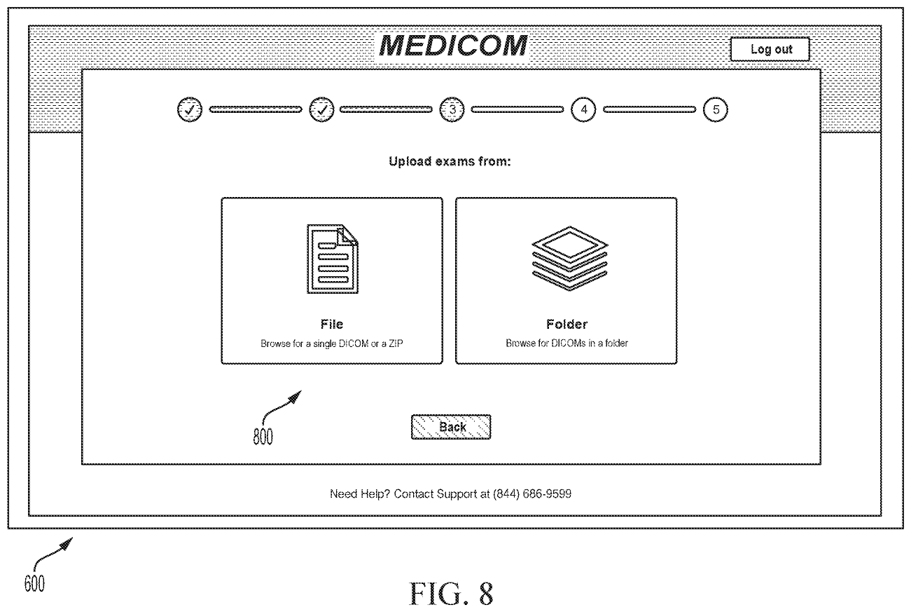

FIG. 8 is an exemplary medical record upload type interface 800 for an uplink portal 600, according to an embodiment of the invention. The medical record upload type interface 800 allows the external provider 128 to select whether to search for individual files, such as a single DICOM file, or to search within folders, such as a folder containing multiple DICOM files.

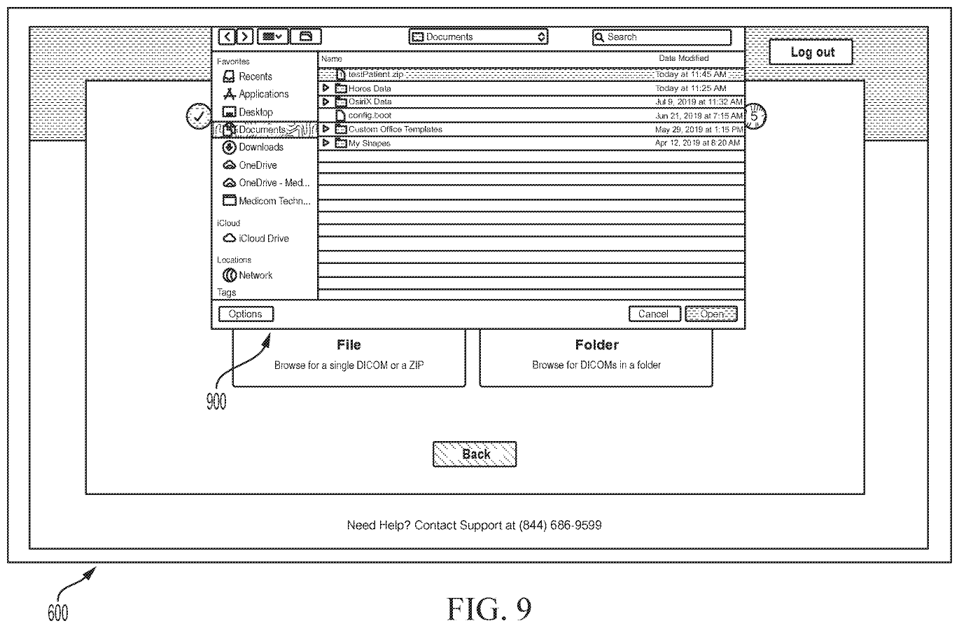

FIG. 9 is an exemplary file browser window 900 for an uplink portal 600, according to an embodiment of the invention. The file browser window 900 allows the external provider 128 to select a file, multiple files, a folder, and/or multiple folders containing external medical records. In another embodiment, the uplink portal 600 can include a drag-and-drop functionality that allows the external provider 128 to drag files or folders from the file browser window 900 directly onto an area of the uplink portal 600.

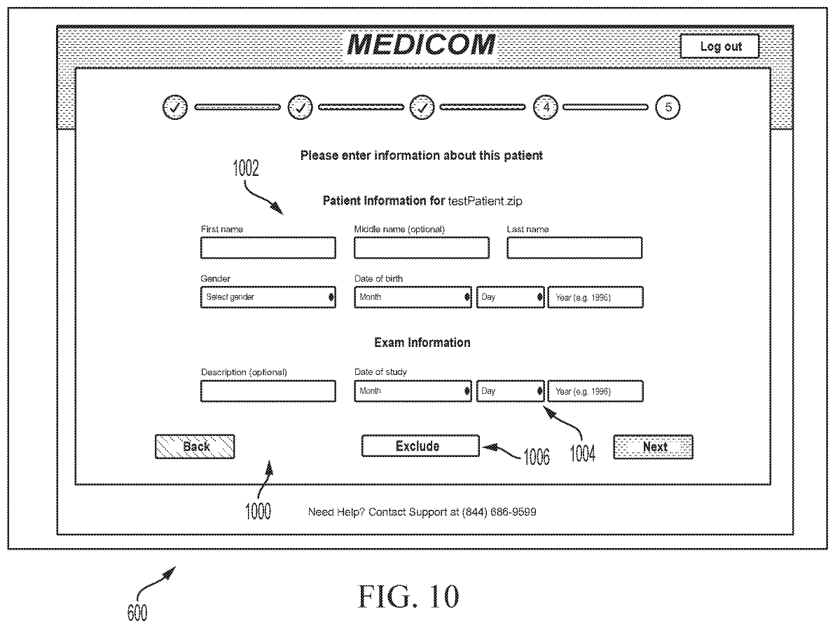

FIG. 10 is an exemplary search interface 1000 for an uplink portal 600, according to an embodiment of the invention. The search interface 1000 allows the external provider 128 to search and filter external medical records based on various criteria 1002. In an embodiment, the external provider 128 can search for a specific patient using criteria 1002 such as a first name, middle name, last name, gender, date of birth, a social security number, an address, a telephone number, a medical record number, or any other unique character string related to the patient whose records are being requested by the diagnostic provider 114. In an embodiment, the external provider 128 can further search and filter based on exam information 1004, such as a visit description, an insurance code, a study date, and the like.

In another embodiment, the search interface can allow additional criteria (not shown), such as a patient age or age range, modality, imaging center, referring physician, date, and the like.

In an embodiment, the search interface 1000 further allows the external provider 128 to specify whether the input criteria 1002 and/or exam information 1006 (collectively, the “input criteria”) should result in external medical records matching the input criteria, or alternatively, should result in external medical records not matching the input criteria. In an embodiment, the external provider 128 can select an “Exclude” option 1006 to specify that the results should exclude external medical records matching the input criteria.

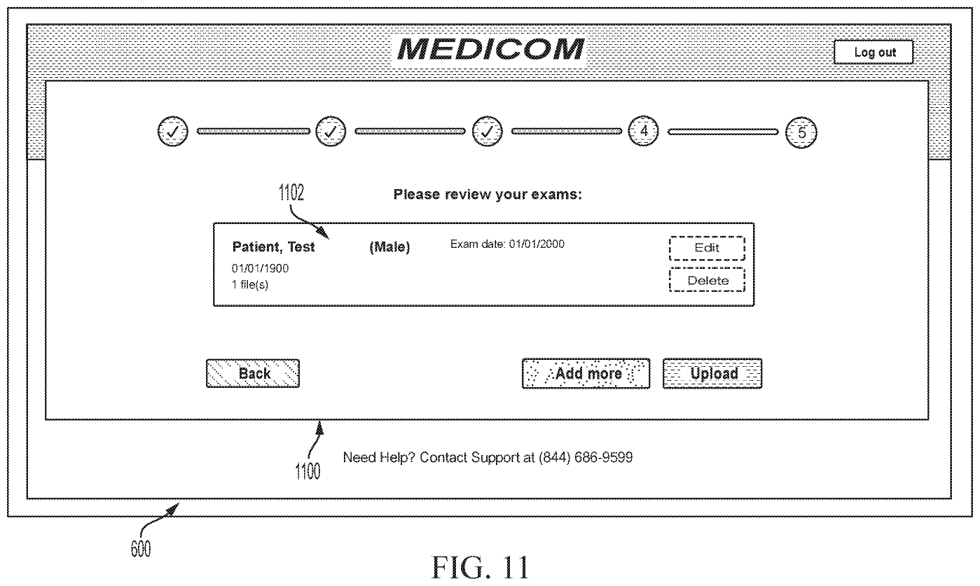

FIG. 11 is an exemplary interface 1100 to confirm a medical record selection for an uplink portal 600, according to an embodiment of the invention. The interface 1100 allows the external provider 128 to confirm a selected external medical record 1102. In an embodiment, the external provider 128 can delete the selected external medical record 1102 from the transmission queue. In addition, the external provider 128 can edit the selected external medical record 1102 to add, remove, and/or replace components of the external medical record. For example, FIG. 11 depicts an “exam date” of Jan. 1, 2000 for the selected external medical record 1102. If the requested patient has additional exam dates, the external provider 128 can edit the selection to include records from an additional exam date.

In an embodiment, the external provider 128 can upload external medical records for multiple patients, in the event the electronic request included a request related to multiple patients.

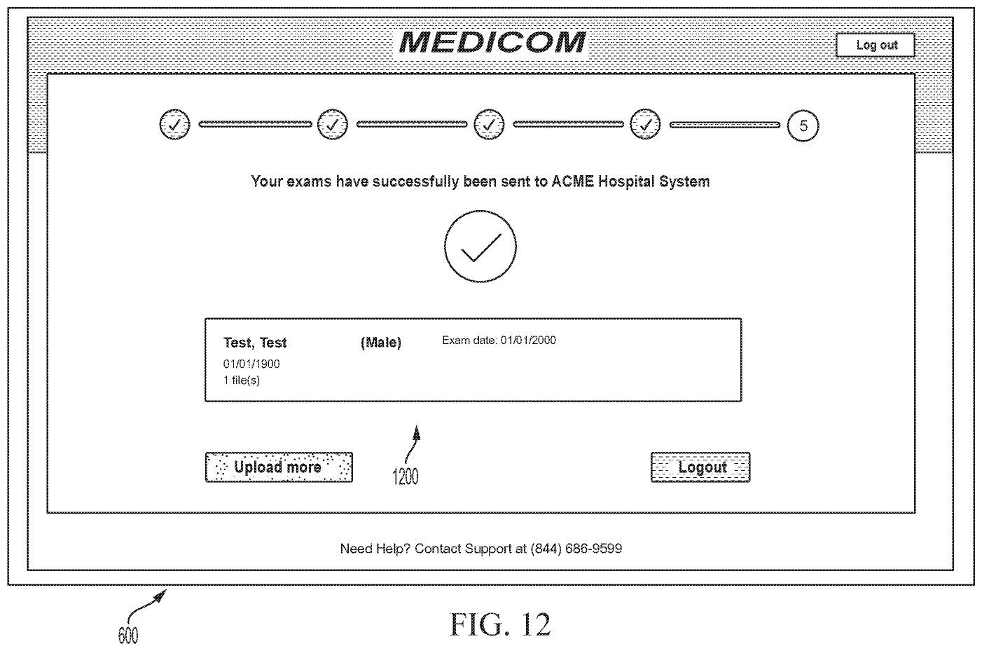

FIG. 12 is an exemplary confirmation screen 1200 for an uplink portal 600, according to an embodiment of the invention. The confirmation screen 1200 can be displayed upon successful transmission of the selected external medical record from the external provider 128 to the diagnostic provider 114. In an embodiment, the confirmation screen 1200 can indicate to the external provider 128 a date and time when the selected external medical record is opened, viewed, or otherwise accessed by the diagnostic provider 114.

As described herein, the patient can utilize the uplink portal in a similar manner as the external provider 128. In an embodiment, the patient can launch the uplink portal via a patient portal software accessed on the patient's computing device. The patient can then upload their medical records to the uplink portal, whereby the uploaded medical records are then securely transmitted to the diagnostic provider 114 via an encrypted peer-to-peer communication channel directly from the patient's computing device.

While the principles of the disclosure have been illustrated in relation to the exemplary embodiments shown herein, the principles of the disclosure are not limited thereto and include any modification, variation or permutation thereof.

What is claimed is:

1. A method for retrieving a medical record from a remote database, comprising:
scheduling a patient for a medical visit by a first medical provider;
determining, by the first medical provider, if the patient has a prior medical record located at a second medical provider;
determining if the first medical provider and the second medical provider are both registered on a common data exchange network;
transmitting, by the first medical provider, an electronic message to the second medical provider if the first medical provider and the second medical provider are not both registered on the data exchange network, wherein the electronic message includes a request for the prior medical record;
transmitting, by the first medical provider, a direct message to the second medical provider via the common data exchange network if the first medical provider and the second medical provider are both registered on the common data exchange network, wherein the in-network message includes a request for the prior medical record;
uploading, by the second medical provider, the prior medical record, to a secure portal; and
transmitting the prior medical record by the second medical provider to the first medical provider via an encrypted peer-to-peer communication channel.

2. The method of claim 1, wherein the electronic message is selected from a group consisting of an electronic facsimile, an electronic mail, and an HL7 message.

3. The method of claim 1, wherein the electronic message and the direct message includes a hyperlink to access the secure portal.

4. The method of claim 3, wherein the secure portal is displayed on a lightweight browser launched upon selection of the hyperlink.

5. The method of claim 3, wherein the secure portal is loaded from a software file that is downloaded upon selection of the hyperlink.

6. The method of claim 1, wherein the secure portal is configured to display a file browser containing a list of medical records in a database associated with the second medical provider.

7. The method of claim 1, wherein the prior medical record includes at least one of a DICOM image and a compressed file.

8. A method for retrieving a medical record from a remote database, comprising:
scheduling a patient for a medical visit by a first medical provider;
determining, by the first medical provider, if the patient has a prior medical record located at a second medical provider;
transmitting, by the first medical provider, an electronic message to the second medical provider, wherein the electronic message includes a request for the prior medical record, a hyperlink to access a secure portal, and a designated storage location;
uploading, by the second medical provider, the prior medical record to a secure portal;
transmitting the prior medical record by the second medical provider to the first medical provider via an encrypted peer-to-peer communication channel; and
receiving the prior medical record at the first medical provider, wherein the prior medical record is stored at the designated storage location.

9. The method of claim 8, wherein the electronic message is selected from a group consisting of an electronic facsimile, an electronic mail, an HL7 message, and a direct message.

10. The method of claim 8, wherein the secure portal is displayed on a lightweight browser launched upon selection of the hyperlink.

11. The method of claim 8, wherein the secure portal is loaded from a software file that is downloaded upon selection of the hyperlink.

12. The method of claim 8, wherein the secure portal is configured to display a file browser containing a list of medical records in a database associated with the second medical provider.

13. The method of claim 8, wherein the prior medical record includes at least one of a DICOM image and a compressed file.

14. The method of claim 8, wherein the designated storage location includes at least two locations where separate components of the prior medical record are respectively stored.

15. A method for retrieving a medical record from a remote database, comprising:
scheduling a patient for a medical visit by a first medical provider, wherein the first medical provider has an internal patient record containing internal patient information;
determining, by the first medical provider, if the patient has an external medical record located at a second medical provider;
transmitting, by the first medical provider, an electronic message to the second medical provider, wherein the electronic message includes a request for the external medical record, a hyperlink to access a secure portal, and a designated storage location;
uploading, by the second medical provider, the external medical record to a secure portal, wherein the external medical record contains external patient information;
transmitting the external medical record by the second medical provider to the first medical provider via an encrypted peer-to-peer communication channel;
receiving the external medical record at the first medical provider, wherein the external medical record stored at the designated storage location; and
updating, by the first medical provider, the external patient record to match the internal patient information.

16. The method of claim 15, wherein the electronic message is selected from a group consisting of an electronic facsimile, an electronic mail, an HL7 message, and a direct message.

17. The method of claim 15, wherein the secure portal is displayed on a lightweight browser launched upon selection of the hyperlink.

18. The method of claim 15, wherein the secure portal is loaded from a software file that is downloaded upon selection of the hyperlink.

19. The method of claim 15, wherein the secure portal is configured to display a file browser containing a list of medical records in a database associated with the second medical provider.

20. The method of claim 15, wherein the designated storage location includes at least two locations where separate components of the external medical record are stored.
